(19) United States
(12) Patent Application Publication
(10) Pub. No.: US 2006/0088583 A1
(43) Pub. Date: Apr. 27, 2006

(54) Artificial oxygen carrier containing preventive agents of metHb formation

(75) Inventors: Shinji Takeoka, Tokyo (JP); Eishun Tsuchida, Tokyo (JP); Hiromi Sakai, Tokyo (JP); Yuji Teramura, Sakai (JP); Tomoyasu Atoji, Mihara-gun (JP)

Correspondence Address: DARBY & DARBY P.C., P. O. BOX 5257, NEW YORK, NY, 10150-5257, US

(73) Assignees: Oxygenix Co., Ltd., Tokyo (JP); Waseda University, Tokyo (JP)

(21) Appl. No.: 11/180,982

(22) Filed: Jul. 12, 2005

(30) Foreign Application Priority Data
Oct. 25, 2004 (JP) 2004-309268

(51) Int. Cl. A61K 38/42; A61K 31/198; A61K 9/127

(52) U.S. Cl. 424/450; 514/6; 514/567

(57) ABSTRACT

The present invention provides an agent containing L-tyrosine that prevents methemoglobin formation, and a vesicle comprising the above agent for preventing methemoglobin formation. More specifically, the present invention provides an oxygen infusion preparation suitable for long-term storage, which prevents an increase in methemoglobin content as a result of oxidation of hemoglobin or the like encapsulated in an vesicle having a lipid bilayer membrane structure.

### FIELD OF THE INVENTION

The present invention relates to an agent containing L-tyrosine that prevents methemoglobin formation, and an artificial oxygen carrier comprising the above agent for preventing methemoglobin formation. More specifically, the present invention relates to an artificial oxygen carrier preparation suitable for long-term storage, which prevents an increase in methemoglobin content as a result of the oxidation of hemoglobin or the like that is encapsulated in a lipid vesicle having a bilayer membrane structure.

### RELATED ART

It has been pointed out that the current blood transfusion system for injecting blood of a suitable blood type into a vein is problematic in the following respects:

(1) there is a possibility of infection (hepatitis, AIDS virus, or the like);

(2) the storage period of red cells is 3 weeks;

(3) with the arrival of an aging society, the number of elderly people among all patients to be treated by blood transfusion increases, while the total number of healthy blood donors is continuously decreasing;

(4) there is a risk of contamination when blood is being stored;

(5) blood transfusion cannot be applied to patients who refuse such treatment for religious reasons;

(6) it is difficult for blood transfusion to respond to urgent demand in disaster situations; and

(7) blood transfusion accidents may occur due to blood type incompatibility.

Thus, an alternative allowing rapid response to demand for transfusion at any time regardless of blood type has been strongly required. As alternatives, conventional infusion preparations such as electrolyte infusions or colloidal infusions have been widely used. However, these infusion preparations offer no alternatives to the most important function of the blood; that is, the function of red cells to carry oxygen. Hence, it has been desired that a substance with an alternative ability to carry oxygen (an oxygen infusion or artificial oxygen carrier) be developed.

The development of an oxygen infusion has also progressed, using hemoglobin having the function of dissociating the binding of oxygen (human hemoglobin, bovine hemoglobin, genetically modified hemoglobin, and the like). Clinical tests regarding intramolecularly crosslinked hemoglobin, water-soluble polymer-binding hemoglobin, intermolecularly crosslinked polymerized hemoglobin, and the like have been conducted in Europe and the United States. In such clinical tests, various types of side effects caused by noncellular structure have been pointed out, and at the same time, the importance of the so-called cellular structure, wherein hemoglobin is encapsulated in a vesicle or capsule, has been clarified.

It was discovered that a phospholipid as a biological component forms a lipid vesicle (liposome). Djordjevich and Miller studied a hemoglobin vesicle using a liposome consisting of phospholipid/cholesterol/fatty acid (Fed. Proc. 36, 567, 1977). Thereafter, several groups, including the group of the present inventors, have made progress in studies regarding such a hemoglobin vesicle.

A hemoglobin vesicle is advantageous in the following respects: (1) hemoglobin can be directly used without modification; (2) viscosity, oncotic pressure, and the degree of oxygen affinity can be controlled to any given values; (3) retention time in the blood can be extended; (4) various types of additives can be encapsulated at high concentrations in the water phase in the vesicle; and the like. Of these respects, the advantage (4) above is particularly important in the present invention. To date, the present inventors have established a method for efficiently preparing a hemoglobin vesicle in their own right, and have obtained a hemoglobin vesicle infusion, the values of the physical properties of which are extremely similar to those of blood. The inventors have confirmed by a test involving administration of the infusion to animals that the above hemoglobin vesicle infusion has excellent ability to carry oxygen (Tsuchida ed. Blood Substitutes Present and Future Perspective, Elsevier, Amsterdam, 1998).

A hemoglobin has 4 hemes. When its heme iron is a bivalent iron (Fe(II)), it can reversibly bind to oxygen. However, when its heme iron becomes an oxidized-type trivalent iron (Fe(III)) (this phenomenon being referred to as methemoglobin formation), the resulting hemoglobin (methemoglobin) cannot bind to oxygen. In addition, superoxide radical anions are generated as a result of such methemoglobin formation from hemoglobin binding to oxygen (oxyhemoglobin), and such superoxide radical anions act as oxidizers, so as to promote generation of methemoglobin. A methemoglobin reduction system and an active oxygen elimination system are present in red cells, and a mechanism for not increasing methemoglobin content functions thereby. However, in the case of a hemoglobin vesicle that uses purified hemoglobin, since all these enzyme systems are eliminated in a step of purifying hemoglobin, oxidation of hemoglobin occurs during the storage and after the administration thereof, thereby resulting in a decrease in the ability to carry oxygen.

In order to inhibit such an oxidation reaction, the following methods have been attempted: (i) a method involving addition of both reductants such as glutathione, homocysteine and/or ascorbic acid, and enzymes for eliminating active oxygen, such as catalase and/or superoxide dismutase (Sakai et al., Bull. Chem. Soc. Jpn., 1994; Takeoka et al., Bioconjugate Chem., 8, 539-544, 1997); (ii) a method, which comprises introducing methylene blue acting as an electron transfer substance into an vesicle membrane, and reducing methemoglobin encapsulated in the vesicle due to electron transfer from NADH that is added to the external water phase in the vesicle (Takeoka et al., Bull. Chem. Soc. Jpn., 70, 1171-1178, 1997); and (iii) a method for reducing methemoglobin by irradiation with the near ultraviolet light (Sakai et al., Biochemistry, 39, 14595-14602, 2000). Moreover, as a method for stably storing a hemoglobin vesicle for a long period of time (shelf storage), a method of completely eliminating oxygen and storing the hemoglobin vesicle in a deoxy form has been attempted (Sakai et al., Bioconjugate Chem, 11, 425-432, 2000).

However, the aforementioned methods for reducing or storing the oxidized hemoglobin vesicle still have room for improvement in respect of the points mentioned below.

First, when the blood is used as a raw material, inactivation of viruses is required in a step of purifying hemoglobin. Thus, as in the case of an albumin preparation, it is also necessary to heat hemoglobin at 60° C. for 10 hours or longer. During this step, a methemoglobin reductase system existing in red cells is denatured and inactivated. In order to use the activity of such an enzyme system, if moderate purification were carried out by the hypotonic hemolysis method, the oxidation rate of the obtained hemoglobin vesicle could be suppressed. However, this makes it difficult to achieve inactivation of viruses. In addition, since the enzyme system is chemically unstable, there are concerns about decreases in the activity thereof during long-term storage.

As stated above, by encapsulating reductants such as glutathione or homocysteine in a hemoglobin vesicle, the formed methemoglobin is reduced, and thus it becomes possible to relatively inhibit an oxidation reaction. However, even when methemoglobin does not exist, such reductants are oxidized through reaction with oxygen in the air and are gradually inactivated (autoxidation). Moreover, methemoglobin formation is promoted by active oxygen species such as superoxide radical anions or hydrogen peroxide generated as a result of the above reaction.

When hemoglobin is stored for a long period of time, methemoglobin formation in a hemoglobin vesicle can be inhibited, only in a hermetically sealed state, by completely eliminating oxygen. However, when such a hemoglobin vesicle is actually used as an oxygen carrier, it is used in the form of oxyhemoglobin wherein oxygen naturally exists. Thus, this method cannot be a means for solving methemoglobin formation in a hemoglobin vesicle. When a hemoglobin vesicle is used as a perfusate for a transplanted organ or as an extracorporeal circulation fluid for example, it is exposed to the atmospheric air for a certain period of time. Thus, the aforementioned methemoglobin formation occurs.

Accordingly, it has been desired to develop a dispersion system, which suppresses the rate of methemoglobin formation in a hemoglobin vesicle in the presence of oxygen, and wherein additives stably exist without reacting with oxygen, differing from a reductant.

### SUMMARY OF THE INVENTION

The present inventors have conducted systematic studies regarding an artificial oxygen carrier over a long period of time. As a result of intensive studies directed towards developing a method for suppressing the rate of methemoglobin formation in a hemoglobin vesicle, the inventors have conceived of the present invention that solves the aforementioned problems.

That is to say, the present invention has the following features:

(1) A method for preventing methemoglobin formation using tyrosine. An example of such tyrosine may be L-tyrosine.

The concentration of L-tyrosine is between 0.01 mM and 20 mM, preferably between 1 mM and 20 mM, and more preferably between 8 mM and 20 mM.

(2) An artificial oxygen carrier comprising a lipid vesicle, in which an agent containing tyrosine that prevents methemoglobin formation and a hemoprotein have been encapsulated.

(3) A method for producing an artificial oxygen carrier, which is characterized in that it comprises encapsulation of an agent containing tyrosine that prevents methemoglobin formation and a hemoprotein in a lipid vesicle.

(4) A method for preventing methemoglobin formation from a hemoprotein, which is characterized in that it comprises encapsulation of an agent containing tyrosine that prevents methemoglobin formation and a hemoprotein in a lipid vesicle.

(5) A method for storing an artificial oxygen carrier, which is characterized in that it comprises encapsulation of an agent containing tyrosine that prevents methemoglobin formation and a hemoprotein in a lipid vesicle.

(6) In the methods described in (3) to (5) above, an example of a hemoprotein may be hemoglobin. In addition, in the methods of the present invention, enzyme species (e.g. catalase, methemoglobin, etc.) can also be encapsulated in a lipid vesicle.

In the present invention, an example of a hemoprotein may be hemoglobin that can reversibly bind to oxygen. In addition, the aforementioned lipid vesicle further comprises enzyme species (e.g. catalase, etc.). Moreover, since methemoglobin also exhibits peroxidase activity having tyrosine as a substrate, it may also be included therein. Furthermore, the aforementioned lipid vesicle is composed of a monolayer or multilayer membrane, and such a membrane of the lipid vesicle may be modified with polyethylene glycol or the like. Still further, in the artificial oxygen carrier and methods of the present invention, when the lipid vesicle, in which an agent containing tyrosine that prevents methemoglobin formation and a hemoprotein have been encapsulated, is left at 37° C. under a partial pressure of oxygen of between 5 and 300 Torr for 60 hours, the rate of methemoglobin is preferably 50% or less. Still further, when hydrogen peroxide is added to the lipid vesicle, in which an agent containing tyrosine that prevents methemoglobin formation and a hemoprotein have been encapsulated, and when the mixture is then left for 60 minutes, the rate of methemoglobin is preferably 20% or less.

The present invention provides a method for preventing methemoglobin formation using tyrosine, and an artificial oxygen carrier comprising a lipid vesicle, in which an agent containing tyrosine that prevents methemoglobin formation and a hemoprotein have been encapsulated. The artificial oxygen carrier of the present invention is able to prevent an increase in methemoglobin content as a result of oxidation of oxyhemoglobin that is encapsulated in a lipid vesicle having a membrane structure. Accordingly, the artificial oxygen carrier of the present invention is useful as an artificial oxygen carrier with a long validated period of the use.

### BRIEF DESCRIPTION OF THE DRAWINGS

FIG. 1 is a view showing a comparison made between the effects of L-Tyr and of D-Tyr to inhibit methemoglobin formation (L-Tyr (◯); D-Tyr (●)). Only L-tyrosine inhibits methemoglobin formation. D-tyrosine does not have such an effect of inhibiting methemoglobin formation. These results show that hemoglobin specifically interacts with L-tyrosine.

FIG. 2 is a view showing the results of an experiment wherein hydrogen peroxide was frequently added to an oxyhemoglobin solution in which methemoglobin and L-tyrosine had previously allowed to coexist, so as to generate methemoglobin. When compared with a control system in which only oxyhemoglobin existed (◯), the system in which methemoglobin and L-tyrosine were allowed to coexist with oxyhemoglobin (●) was significantly inhibited in terms of an increase in the rate of methemoglobin. A system in which only L-tyrosine was added to oxyhemoglobin (□) exhibited almost the same behavior as that of the above control system in terms of an increase in the rate of methemoglobin. In a system in which only methemoglobin was added to oxyhemoglobin (▪), methemoglobin formation was promoted by side reactions (Fenton's reaction and the like) caused by the release of iron ions due to denaturation of methemoglobin caused by hydrogen peroxide added. These results show that hydrogen peroxide is eliminated by the peroxidase activity of methemoglobin having L-tyrosine as a substrate.

FIG. 3 is a view showing successive addition of hydrogen peroxide to a hemoglobin vesicle, in which high concentrations of methemoglobin and L-tyrosine have been encapsulated.

### DESCRIPTION OF THE PREFERRED EMBODIMENTS

The present invention will be described in detail below. The following embodiments are provided for illustrative purposes only, and are not intended to limit the scope of the invention.

All publications, patents, and patent applications cited herein are incorporated herein by reference in their entirety.

The present invention has been completed based on the properties of tyrosine (in particular, L-tyrosine) to prevent methemoglobin formation. Thus, the present invention relates to application of tyrosine to an artificial oxygen carrier or an agent for preventing the blood from undergoing methemoglobin formation. The term “methemoglobin formation” is used herein to mean the oxidization of the center iron of protoheme as a prosthetic group of hemoglobin, followed by its conversion from bivalent iron (Fe²⁺) to trivalent iron (Fe³⁺).

The purpose of use of tyrosine is not particularly limited herein, as long as it is used to prevent methemoglobin formation in red cells (including prevention of an increase in methemoglobin formation). Examples of such purpose of use of tyrosine may include: dilution of the blood before operation; extracorporeal circulation; organ preservation; liquid ventilation; the treatment of sickle cell anemia, apoplexy, carbon monoxide intoxication, cancers, or toxicosis associated with deglutition; and other clinical treatments. However, examples are not limited thereto.

In order to use tyrosine for the aforementioned purposes, tyrosine can be encapsulated in a lipid vesicle such as a liposome.

The term “lipid vesicle” is used in the present invention to mean the molecular assembly of vesicle structures having membranes, which are constituted by the interaction (hydrophobic interaction, electrostatic interaction, hydrogen bond, etc.) between the molecules of a lipid and/or a lipoprotein in an aqueous solvent, without involving a covalent bond. The above membrane constitutes a monolayer or multilayer (a bilayer, for example).

The lipid vesicle used in the present invention can be comprised of phospholipids alone or in combination with cholesterols or fatty acids. Such a vesicle can be prepared by the method that the present inventors have previously disclosed (Sakai et al., Biotechnol. Progress, 12, 119-125, 1996; Bioconjugate Chem., 8, 23-30, 1997). Specifically, as allosteric factors, appropriate amounts of pyridoxal 5′-phosphate and L-tyrosine are first added to a purified hemoglobin solution, and mixed lipid powders are also added thereto, followed by hydration. Using a high pressure extruder, the thus obtained hemoglobin-lipid mixed solution is permeated stepwise through filters with pore sizes ranging from 3 μm to 0.22 μm, so as to regulate particle diameter. Thereafter, unencapsulated hemoglobin portions are eliminated by centrifugation, so as to prepare a hemoglobin vesicle.

In the present invention, other than the aforementioned method, common methods for producing an vesicle, such as ultrasonic irradiation, forced stirring (homogenizer) method, vortex mixing method, freezing and thawing method, organic solvent injection method, surfactant elimination method, reverse phase evaporation method, or microfluidizer method, can be adopted. For example, the freezing and thawing method comprises: adding pyridoxal 5′-phosphate and L-tyrosine to a purified hemoglobin solution; mixing mixed lipid powers therein, followed by hydration; and repeating freezing (−197° C.) and thawing (40° C.) operations 3 times, so as to prepare a hemoglobin vesicle. The organic solvent injection method comprises: dissolving mixed lipids in chloroform or a mixed solvent consisting of diethyl ether and methanol; injecting the obtained solution into a purified hemoglobin solution, to which pyridoxal 5′-phosphate and L-tyrosine have been added; and eliminating the solvent by pressure reduction, so as to prepare a hemoglobin vesicle. The ultrasonic irradiation method comprises: adding pyridoxal 5′-phosphate and L-tyrosine to a purified hemoglobin solution; mixing mixed lipid powers therein, followed by hydration; and applying ultrasound to the obtained solution using a probe-type ultrasonic irradiation device, so as to prepare a hemoglobin vesicle.

Either a saturated phospholipid or a unsaturated phospholipid may be used as a phospholipid that is a constitutional component of the aforementioned vesicle (Japanese Patent No. 2936109). Examples of a phospholipid used herein may include egg-yolk lecithin, hydrogenated lecithin, dimyristoyl phosphatidylcholine, dipalmitoyl phosphatidylcholine, distearoyl phosphatidylcholine, dioleoyl phosphatidylcholine, dilinoleoyl phosphatidylcholine, phosphatidic acid, phosphatidylethanolamine, phosphatidylglycerol, and phosphatidylinositol. These phospholipids can be selected from among polymerizable phospholipids having a polymerizable group such as -ene (double bond), -yne (triple bond), diene, diyne, or styrene. Examples of such a polymerizable phospholipid may include 1,2-di(octadeca-trans-2,trans-4-dienoyl) phosphatidylcholine, 1,2-di(octadeca-2,4-dienoyl)phosphatidic acid, and 1,2-bis-eleostearoyl phosphatidylcholine. As fatty acid, a saturated or unsaturated fatty acids having 12 to 20 carbon atoms is used. Examples of such fatty acid may include myristic acid, palmitic acid, stearic acid, oleic acid, linoleic acid, linolenic acid, and octadeca-2,4-dienoic acid.

In the present invention, suitable additives may be added to the membrane of the aforementioned molecular assembly of lipid, so as to modify the membrane. Examples of such an additive used for modifying the membrane may include sialic acid, sugar-binding fatty acid, polyoxyethylene-binding phospholipid, and polyoxyethylene-binding fatty acid. Preferably, the membrane is modified with polyoxyethylene (polyethylene glycol). The molecular weight of polyethylene glycol is between approximately 400 Da and 12,000 Da, and preferably between 1,000 Da and 5,000 Da.

Examples of a hemoprotein encapsulated in the aforementioned lipid vesicle may include hemoglobin, myoglobin, and albumin-heme. A purified hemoglobin can be produced by methods known in the present field (edited by the Japanese Biochemical Society, Zoku-Seikagaku Jikken Koza, Vol. 8, “Ketsueki (Blood),” No. 1, Tokyo Kagaku Dojin Co., Ltd., 1987; Methods in Enzymology, Volume 76, 1981, Academic Press, New York; The Chromatograph of Hemoglobin, 1983, Dekker, New York; etc.). When hemoglobin is purified by the hemolysis method, for example, a hypotonic solution is added to washed red cells, the blood is then hemolyzed by the difference in osmotic pressures, and thereafter, red cell membrane components are eliminated by centrifugation. Thereafter, ultrafiltration, crystallization, or HPLC is performed on the resultant, so as to obtain highly purified hemoglobin.

Moreover, carbon monoxide is allowed to bind to hemoglobin (HbCO), so as to suppress methemoglobin formation and also so as to improve high-temperature stability. In this case, this means is effective for completely eliminating remaining solvents that have been used for purification by a treatment with solvents (for example, carbon tetrachloride, toluene, chloroform, diethyl ether, or the like). Proteins existing with hemoglobin can be eliminated by heating. Since HbCO is stable against heating, it can inactivate contaminant proteins or coexisting viruses.

In the present invention, a vesicle in which hemoglobin has been encapsulated as a water-soluble substance is referred to as a “hemoglobin vesicle.” Hereafter, a hemoglobin vesicle in which L-tyrosine has been encapsulated will be described as an example. However, examples are not limited thereto.

When L-tyrosine is applied in the present invention, the L-tyrosine has preferably been encapsulated in a hemoglobin vesicle. It is possible that L-tyrosine encapsulated in the hemoglobin vesicle of the present invention be mixed in a water-soluble substance after preparation of the hemoglobin vesicle. However, in order for the hemoglobin vesicle to suppress methemoglobin formation at a high rate, it is preferable that L-tyrosine has previously been added to a water-soluble substance (dispersion), when such a hemoglobin vesicle is prepared. In the present invention, it is preferable to use L-tyrosine in the form of a monomer.

Moreover, with an increase in the concentration of L-tyrosine, the effect of such a hemoglobin vesicle to suppress methemoglobin formation increases. Accordingly, the higher the concentration of L-tyrosine added as an agent for preventing methemoglobin formation, the better the effects that can be obtained in the present invention. In the present invention, the additive amount of L-tyrosine is at least 0.01 mM, preferably 1.0 mM or more, and more preferably 8.0 mM or more. At maximum, approximately 20 mM L-tyrosine can be dissolved, for example.

When the agent for preventing methemoglobin formation of the present invention is used, a dispersion of a hemoglobin vesicle is diluted with a saline solution to a certain component concentration (for example, hemoglobin concentration: 5 g/dL). At this time, although such a hemoglobin vesicle dispersion is diluted, the component concentration of the water phase in the vesicle is maintained as is, without being diluted. This is extremely advantageous for application of the method of the present invention. With the assumption that a hemoglobin vesicle containing L-tyrosine is used as an extracorporeal circulation fluid or tissue culture solution, the hemoglobin vesicle containing L-tyrosine is stirred at 37° C. in the atmospheric air or under a low partial pressure of oxygen, so that the rate of methemoglobin formation in the above hemoglobin vesicle containing L-tyrosine can be suppressed when compared with that in a hemoglobin vesicle containing no L-tyrosine. The term “under a low partial pressure of oxygen (low oxygen partial pressure conditions)” is used herein to mean a partial pressure of oxygen of between 5 and 300 Torr, and preferably of 40 Torr, at 37° C.

As mentioned above, since L-tyrosine used in the present invention is able to suppress the rate of methemoglobin formation in a hemoglobin vesicle, it is able to extend the period for the hemoglobin vesicle to function as an oxygen carrier, for a long period of time. For example, when the aforementioned hemoglobin vesicle is used for various types of applications, such as a blood diluent, an extracorporeal circulation fluid, or a tissue culture solution, the rate of methemoglobin formation is suppressed, and thus the period for the hemoglobin vesicle to function as an oxygen carrier can be significantly extended. In addition, by applying the aforementioned hemoglobin vesicle to a method for storing a hemoglobin vesicle in an oxy state, an increase in the concentration of methemoglobin can be suppressed over a long period of time.

As described above, the hemoglobin vesicle of the present invention, in which L-tyrosine has been encapsulated, enables suppression in the rate of methemoglobin formation.

It has been known that when hemoglobin that is in an oxy state is oxidized to methemoglobin, hydrogen peroxide is generated, and that such hydrogen peroxide promotes methemoglobin formation. The recent studies of the present inventors have revealed that when L-tyrosine is specifically oxidized to dityrosine, methemoglobin has enzymatic activity of consuming hydrogen peroxide, namely, peroxidase activity. At present, it is considered that the concentration of hydrogen peroxide in a system is decreased by such activity, and that as a result, methemoglobin formation caused by hydrogen peroxide is suppressed.

Accordingly, if an appropriate amount of methemoglobin has previously been encapsulated in a vesicle containing L-tyrosine and hemoglobin that is in an oxy state, methemoglobin formation from the hemoglobin that is in an oxy state can significantly be suppressed.

Further, it is considered that L-tyrosine does not directly interact with hemoglobin. Actually, when the heat of binding generated as a result of the interaction (binding) of L-tyrosine with hemoglobin was measured by the isothermal titration microcalorimetry method, almost no heat of binding was observed. From the oxygen dissociation curve of hemoglobin to which L-tyrosine was mixed, no particular influence upon the allosteric effect was found, and no change in the degree of oxygen affinity was observed.

Furthermore, various types of enzymes can be encapsulated in the hemoglobin vesicle of the present invention. Examples of such enzymes may include catalase and superoxide dismutase. The additive amount of such enzyme is between 10,000 and 50,000 unit/ml in the case of catalase. It is between 1,000 and 10,000 unit/ml in the case of superoxide dismutase. When the above enzymes are used within the aforementioned ranges of additive amounts, they effectively act to suppress methemoglobin formation.

When a hemoglobin solution containing L-tyrosine and catalase was compared with a hemoglobin solution containing catalase in terms of the rate of methemoglobin formation, the former had a higher effect of suppressing the rate of methemoglobin formation. This is because catalase has high ability to eliminate hydrogen peroxide. For example, hydrogen peroxide was added to a lipid vesicle, in which an agent for preventing methemoglobin formation and a hemoprotein have been encapsulated, and the mixture was then left for 60 minutes. 60 minutes later, the rate of methemoglobin was found to be 20% or less (refer to Examples). Even 420 minutes later, the rate of methemoglobin was found to be 40% or less.

A hemoglobin solution containing L-tyrosine was stirred at 37° C., and it was then analyzed by UV-vis spectrum measurement, fluorometry, and HPLC. As a result, a slight amount of dityrosine was confirmed. Thereafter, this experiment was performed on a mixture obtained by adding hydrogen peroxide to a methemoglobin solution containing L-tyrosine. As a result, a large amount of dityrosine was confirmed. This is because of the peroxidase activity of methemoglobin. Thereafter, the change in methemoglobin concentration was observed during chilled storage (4° C.). A hemoglobin vesicle containing L-tyrosine ([L-tyrosine]=1 mM) (encapsulated system) was compared with an unencapsulated system (wherein, in both systems, the rate of methemoglobin was found to be 3.0%, when they were prepared). 1 month later, the rate of methemoglobin in both systems were found to be 4.4% and 9.3%, respectively. 3 months later, they were found to be 10.2% and 24.3%, respectively. Thus, it was found that methemoglobin formation was significantly suppressed in the encapsulated system. These results show that a hemoglobin vesicle can stably be stored for a long period of time.

As stated above, according to the present invention, the rate of methemoglobin formation is suppressed in the hemoglobin vesicle containing L-tyrosine, thereby extending the period for carrying oxygen.

Moreover, in the present invention, tyrosine is added to a suitable buffer solution, and the obtained mixture can be used as an injection preparation (a liquid preparation used for intravenous, intra-arterial or subcutaneous injection, or a liquid preparation used for extracorporeal treatment). It is also possible to add various types of additives to the aforementioned preparation. Examples of such an additive may include a preservative, a buffer, and a solvent.

In the present invention, when tyrosine is added to a patient for the purpose of clinical medicine, the dosage of an active ingredient thereof is between 100 μg/kg and 1,000 mg/kg, and preferably between 500 μg/kg and 10 mg/kg, per day.

### EXAMPLES

The present invention will be more specifically described below in the following examples. However, these examples are not intended to limit the scope of the present invention.

### Example 1

Preparation of Hemoglobin Vesicle Containing L-Tyrosine and Autoxidation in the Atmospheric Air (37° C.)

In an aseptic atmosphere, pyridoxal 5′-phosphate (PLP, [PLP]/[Hb]=2.5) as an allosteric factor and L-tyrosine were added to a highly purified stroma-free hemoglobin solution (36 g/dL) obtained by purification of human red cells derived from the donated blood, resulting in the concentration of L-tyrosine of 50, 100, 250, and 500 μM. Otherwise, such components were not added to the above hemoglobin solution. Thereafter, using Remolino™ (manufactured by Millipore Japan), each of the obtained mixtures was filtrated through an FM microfilter with a pore size of 0.22 μm (manufactured by Fuji Photo Film Co., Ltd.), so as to obtain a processed hemoglobin solution. Mixed lipid powders (a mixture consisting of phosphatidylcholine, cholesterol, and DPEA; manufactured by Nippon Fine Chemical) were added, little by little, to the hemoglobin solution, resulting in the concentration of lipid of 4.5 wt %. The mixture was then stirred at 4° C. for 12 hours, so as to obtain a multilayer vesicle, in which hemoglobin had been encapsulated. The particle diameter and the number of coating layers were regulated by the extrusion method using Remolino. The FM microfilters were used in the order of pore sizes of 3, 0.8, 0.65, 0.45, 0.3, and 0.22 μm. The obtained hemoglobin vesicle dispersion was diluted with a saline solution. The diluted solution was subjected to ultracentrifugation (50,000 g, 40 minutes), and the supernatant hemoglobin solution was then eliminated by aspiration. Thereafter, a polyoxyethylene-binding lipid [N-(monomethoxy polyethylene glycol-carbamyl)distearoyl phosphatidyl ethanolamine; the molecular weight of the polyethylene glycol chain: 5,300] dispersed in a saline solution was added thereto, at an amount corresponding to 0.3 mol % of the lipid on the outer surface of the vesicle. The mixture was stirred at 25° C. for 2 hours, so as to modify the surface of the hemoglobin vesicle with polyethylene glycol. The concentration of hemoglobin was set at 10 g/dL, and the mixed solution was then filtrated through a 0.45-μm filter (Dismic-25; ADVANTEC), so as to obtain a polyethylene glycol-modified hemoglobin vesicle.

A dispersion of the hemoglobin vesicle containing L-tyrosine ([L-tyrosine]=50, 100, 250, and 500 μM) or the hemoglobin vesicle was stirred at 37° C. in the atmospheric air. Each sample was collected over time. Thereafter, the rate of methemoglobin was calculated from the ratio of the absorbance of the hemoglobin vesicle solution at 405 nm to that of at 430 nm. As a result, it was found that as the concentration of L-tyrosine added increases, the rate of methemoglobin formation in the hemoglobin vesicle containing the L-tyrosine is suppressed. When the time at which the rate of methemoglobin becomes 50% was defined as T1/2, such T1/2 was 18 hours in the case of a hemoglobin vesicle containing no L-tyrosine. In contrast, in the case of hemoglobin vesicles containing L-tyrosine with a concentration of 50, 100, 250, or 500 μM, such T1/2 were 20, 24, 27, and 30 hours, respectively. Thus, T1/2 was drastically extended by the presence of L-tyrosine.

### Example 2

Autoxidation of L-Tyrosine-Containing Hemoglobin Vesicle Under a Partial Pressure of Oxygen of 40 Torr (37° C.)

A dispersion of the L-tyrosine-containing hemoglobin vesicle ([L-tyrosine]=50, 100, 250, and 500 μM) or a hemoglobin vesicle prepared in Example 1 was stirred at 37° C. under a partial pressure of oxygen of 40 Torr. Thereafter, each sample was collected over time. Thereafter, the rate of methemoglobin was calculated from the absorbance ratio. As a result, it was found that as the concentration of L-tyrosine added increases, the rate of methemoglobin formation in the L-tyrosine-containing hemoglobin vesicle is suppressed. The time T1/2 at which the rate of methemoglobin becomes 50% was 12.5 hours in the case of a hemoglobin vesicle containing no L-tyrosine. In contrast, in the case of hemoglobin vesicles containing L-tyrosine with a concentration of 50, 100, 250, or 500 μM, such T1/2 were 14, 15, 16, and 18.5 hours, respectively. Thus, T1/2 was drastically extended by the presence of L-tyrosine.

### Example 3

Autoxidation of L-Tyrosine-Containing Hemoglobin Vesicle in the Atmospheric Air (4° C.)

A dispersion of the L-tyrosine-containing hemoglobin vesicle ([L-tyrosine]=1 mM) or a hemoglobin vesicle prepared in Example 1 was stored at 4° C. in the atmospheric air. Each sample was collected over time. Thereafter, the rate of methemoglobin was calculated from the absorbance ratio. As a result, it was found that as the concentration of L-tyrosine added increases, the rate of methemoglobin formation in the L-tyrosine-containing hemoglobin vesicle is suppressed. The rate of methemoglobin of the hemoglobin vesicle containing L-tyrosine and that of the hemoglobin vesicle containing no L-tyrosine were both 3.0%, when they were prepared. 1 month later, the rate of methemoglobin were 4.4% and 9.3%, respectively. 3 months later, they were 10.2% and 24.3%, respectively. Thus, significant suppression in the rate of methemoglobin formation was observed in the hemoglobin vesicle containing L-tyrosine.

### Example 4

Measurement of the Time Required for Methemoglobin Formation Using Vesicle Containing Each of Tyrosine Derivative, Antioxidant, and Phenol Derivative

A hemoglobin vesicle dispersion containing each of a tyrosine derivative, various types of antioxidants, and various types of phenol derivatives, at certain concentrations, was produced. Thereafter, the rate of methemoglobin formation was measured in the same manner as in Example 1.

The results are shown in Table 1. The following Table 1 shows the initial rate of methemoglobin formation using each of the tyrosine derivative, antioxidants, and phenol derivatives, and the time required for 50% methemoglobin formation. From the results, it was found that L-tyrosine most effectively suppresses methemoglobin formation in a hemoglobin vesicle.

TABLE 1
Initial rate of methemoglobin formation using tyrosine,
antioxidants and phenol derivatives, and time required
for 50% methemoglobin formation

| | | Initial rate | Time required |
|---|---|---|---|
| | | of metHb | for 50% metHb |
| | Concentration | formation | formation |
| | (mM) | (% hr) | (hr) |
| Control | | 1.3 | 34.0 |
| L-tyrosine | 0.25 | 1.1 | 42.0 |
| | 0.5 | 1.0 | 48.0 |
| | 1 | 0.9 | 52.0 |
| (1) Flavonoid antioxidants | | | |
| Kaempferol | 1 | 3.1 | 25.0 |
| Apigenin | 0.1 | 1.6 | 34.8 |
| | 1 | 1.4 | 35.6 |
| (2) Catechin antioxidants | | | |
| Epigallocatechin gallate | 1 | 3.4 | 12.4 |
| | 2 | 3.2 | 13.6 |
| | 3 | 3.2 | 13.0 |
| (3) Phenol derivatives | | | |
| Phenol | 0.1 | 1.1 | 35.6 |
| | 1 | 1.3 | 33.2 |
| p-hydroxyphenyl acetic | 0.1 | 1.5 | 30.6 |
| acid | 1 | 1.7 | 30.2 |
| | 3 | 1.8 | 28.3 |
| 3-(p-hydroxy- | | 0.9 | 34.7 |
| phenyl)pronionic acid | 1 | 1.0 | 32.2 |
| | 3 | 1.2 | 34.2 |
| 3,4-dihydroxyphenol- | 0.1 | 1.4 | 30.8 |
| L-alanine | 1 | 3.4 | 14.4 |
| | 3 | 5.3 | 8.2 |

### Example 5

Methemoglobin Formation Suppression Test Using L-Tyrosine and D-Tyrosine

A hemoglobin vesicle dispersion, in which an oligopeptide containing D-tyrosine or L-tyrosine had been encapsulated, was prepared. Thereafter, the rate of 50% methemoglobin formation was measured in the same manner as in Example 1.

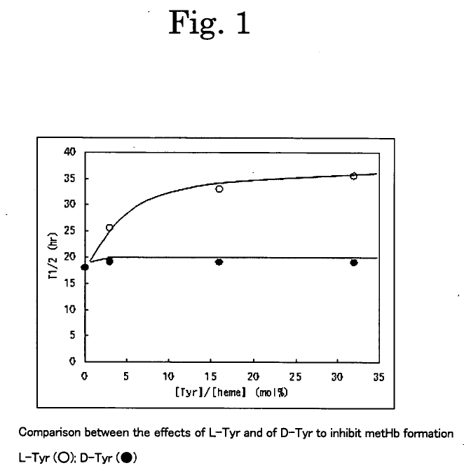

As a result, the effectiveness of L-tyrosine for suppression of methemoglobin formation was confirmed (FIG. 1).

### Example 6

Methemoglobin Formation Suppression Test Using Enzymes in Combination (1)

A hemoglobin vesicle dispersion containing 0.25 ml of L-tyrosine, another hemoglobin vesicle dispersion containing 50,000 units/ml catalase, and another hemoglobin vesicle dispersion containing both 0.25 ml of L-tyrosine and 50,000 units/ml catalase, were prepared. Thereafter, the time required for 50% methemoglobin formation was measured in the same manner as in Example 1.

The results are shown in Table 2.

TABLE 2
Tyrosine derivatives and time required for 50% metHb formation

| | | | Time required for 50% |
|---|---|---|---|
| | Concentration | | metHb formation (hr) |
| Control | | | 34.0 |
| L-tyrosine | 0.25 | mM | 42.0 |
| Catalase | 50000 | unit/mL | 45.0 |
| L-tyrosine + catalase | 0.25 mM, 50000 unit/mL | | 49.0 |
| L-Tyr-L-Tyr | 0.1 | mM | 32.0 |
| | 0.25 | mM | 32.0 |
| L-Tyr-L-Glu | 0.1 | mM | 33.0 |
| | 0.25 | mM | 33.0 |

The above Table 2 shows the effect of tyrosine to suppress methemoglobin formation, and the effect of catalase to suppress methemoglobin formation. When compared with a control (addition of neither L-tyrosine nor catalase), L-tyrosine significantly suppressed the rate of methemoglobin. Such suppression in the rate of methemoglobin was further enhanced by addition of catalase.

### Example 7

Methemoglobin Formation Suppression Test Using Enzymes in Combination (2)

0.5 wt % methemoglobin/1 mM L-tyrosine was added to a 5 g/dL hemoglobin solution that was in an oxy state ([hemoglobin]=775 μM). Thereafter, 310 μM hydrogen peroxide (the same concentration as that of heme in methemoglobin) was added to the above solution, every 10 minutes, for 60 minutes in the atmospheric air at 37° C., while stirring. 300 μl of a sample was collected immediately before addition of each hydrogen peroxide, and 20 μl of catalase (5,000 units) was promptly added to each sample, so as to eliminate the hydrogen peroxide. Thereafter, the rate of methemoglobin was calculated by the cyanomethemoglobin method.

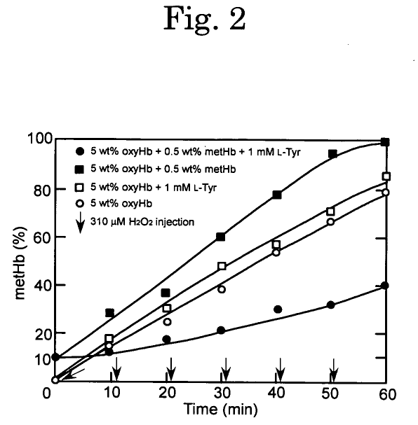

The results are shown in FIG. 2. In a system wherein hydrogen peroxide was added to the oxyhemoglobin solution or the mixed solution consisting of oxyhemoglobin and L-tyrosine, as the number of addition increased, the rate of methemoglobin linearly increased, and it reached approximately 80% for 60 minutes. In a mixed solution consisting of oxyhemoglobin and methemoglobin, promotion in methemoglobin formation caused by the denaturation of methemoglobin due to the added hydrogen peroxide was observed. 60 minutes later, the oxyhemoglobin became 100% methemoglobin. On the other hand, in a mixed solution consisting of oxyhemoglobin, methemoglobin, and L-tyrosine, an increase in the rate of methemoglobin was extremely slow, and 60 minutes later, it was only 40%. The rate of oxyhemoglobin that became methemoglobin was only 30%. From these results, it was confirmed that methemoglobin stably eliminates hydrogen peroxide in the coexistence of L-tyrosine, as with catalase, and that it suppresses methemoglobin formation from oxyhemoglobin.

### Example 8

Preparation of Hemoglobin Vesicle Containing High Concentrations of L-Tyrosine and Methemoglobin

In an aseptic atmosphere, pyridoxal 5′-phosphate (PLP, [PLP]/[Hb]=2.5) as an allosteric factor was added to a highly purified stroma-free hemoglobin solution (36 g/dL) obtained by purification of human red cells derived from the donated blood. Thereafter, a methemoglobin solution was produced by forming methemoglobin using potassium ferricyanide and then eliminating the potassium ferricyanide by gel permeation chromatography. The obtained methemoglobin solution was concentrated to 36 wt % by ultrafiltration. The concentrated methemoglobin solution was added to the above hemoglobin solution to a final concentration of 4 wt %. Thereafter, L-tyrosine was further added thereto to a concentration of 8.5 mM. Otherwise, such components were not added to the above hemoglobin solution. Thereafter, using Remolino™ (manufactured by Millipore Japan), the obtained mixture was filtrated through an FM microfilter with a pore size of 0.22 μm (manufactured by Fuji Photo Film Co., Ltd.), so as to obtain a processed hemoglobin solution. As mixed lipid powders, a mixture having the composition consisting of dipalmitoyl phosphatidylcholine, cholesterol, 1,5-O-dihexadecyl-N-succinyl-L-glutamate, and N-(monomethoxy polyethylene glycol-carbamyl)distearoyl phosphatidyl ethanolamine, was used (manufactured by Nippon Fine Chemical). The molecular weight of the polyethylene glycol chain was 5,300. The above mixed lipid powders were added, little by little, to the above hemoglobin solution, resulting in a concentration of lipid of 4.5 wt %. The mixture was then stirred at 4° C. for 12 hours, so as to obtain a multilamellar vesicle, in which hemoglobin had been encapsulated. The particle diameter and the number of coating layers were regulated by the extrusion method using Remolino™. The FM microfilters were used in the order of pore sizes of 3, 0.8, 0.65, 0.45, 0.3, and 0.22 μm. The obtained hemoglobin vesicle dispersion was diluted with a saline solution. The diluted solution was subjected to ultracentrifugation (50,000 g, 40 minutes), and the supernatant hemoglobin solution was then eliminated by aspiration. Thereafter, the concentration of the resultant hemoglobin was set at 10 g/dL, and the mixed solution was then filtrated through a 0.45-μm filter (Dismic-25; ADVANTEC), so as to obtain a polyethylene glycol-modified hemoglobin vesicle.

### Example 9

Autoxidation of Hemoglobin Vesicle Containing High Concentration of L-Tyrosine Under a Partial Pressure of Oxygen of 40 Torr (37° C.)

A dispersion of the hemoglobin vesicle containing L-tyrosine and methemoglobin ([L-tyrosine]=8.5 mM) or a hemoglobin vesicle prepared in Example 1 was stirred at 37° C. under a partial pressure of oxygen of 40 Torr. Thereafter, each sample was collected over time. Thereafter, the rate of methemoglobin was calculated from the absorbance ratio. As a result, it was found that the rate of methemoglobin reached 20% after approximately 18 hours, and that it reached 50% after 60 hours. In the case of a hemoglobin vesicle containing neither L-tyrosine nor methemoglobin, the rate of methemoglobin became 50% after approximately 13 hours under the same conditions. Thus, it was found that the hemoglobin vesicle containing high concentrations of L-tyrosine and methemoglobin has a significant effect of suppressing the rate of methemoglobin formation.

### Example 10

Successive Addition of Hydrogen Peroxide to Hemoglobin Vesicle Containing High Concentration of L-Tyrosine

Hydrogen peroxide (310 μM, the same concentration as that of encapsulated methemoglobin) was added, every 10 minutes, to the dispersion of the 5 wt % oxyhemoglobin vesicle containing L-tyrosine and methemoglobin ([L-tyrosine]=8.5 mM) or 5 wt % oxyhemoglobin vesicle prepared in Example 8 (at 37° C., in the atmospheric air).

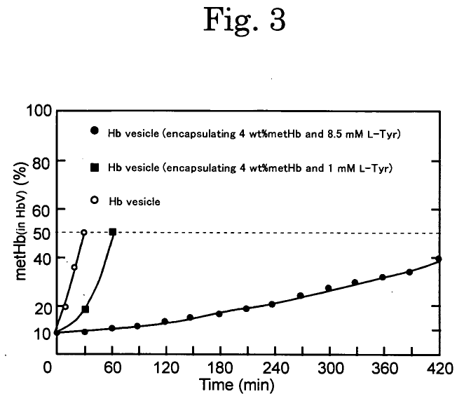

The measurement results are shown in FIG. 3. As shown in FIG. 3, when hydrogen peroxide was added to the 5 wt % oxyhemoglobin vesicle dispersion every 10 minutes, the rate of methemoglobin reached 50% after 30 minutes ((◯) in FIG. 3). In contrast, when hydrogen peroxide was added, every 10 minutes, to a 5 wt % hemoglobin vesicle dispersion, wherein methemoglobin made up 4 wt % of 40 wt % hemoglobin encapsulated in the above vesicle and in which 1 mM L-tyrosine was also encapsulated, the rate of methemoglobin reached 50% after 60 minutes ((▪) in FIG. 3). Thus, this case exhibited approximately 2 times of the extension effect. When 8.5 mM L-tyrosine was encapsulated therein, the rate of methemoglobin reached only 40%, even 420 minutes after addition of hydrogen peroxide ((●) in FIG. 3). These results show that encapsulation of a high concentration of L-tyrosine brings on a significant increase in the above effect.

### INDUSTRIAL APPLICABILITY

A dispersion containing a hemoglobin vesicle can be widely used in the medical and pharmaceutical fields. In particular, by adding various additives to the dispersion, the obtained mixture can be used as an alternative to the blood in the clinical medicine.

1. A method for preventing methemoglobin formation using tyrosine.

2. The method for preventing methemoglobin formation according to claim 1, wherein the tyrosine is L-tyrosine.

3. The method for preventing methemoglobin formation according to claim 2, wherein the concentration of the L-tyrosine is between 0.01 mM and 20 mM.

4. An artificial oxygen carrier comprising a lipid vesicle, which encapsulates an agent containing tyrosine that prevents methemoglobin formation and a hemoprotein.

5. The artificial oxygen carrier according to claim 4, wherein the hemoprotein is hemoglobin.

6. The artificial oxygen carrier according to claim 4, further comprising enzyme species in said vesicle.

7. The artificial oxygen carrier according to claim 6, wherein the enzyme species is catalase.

8. The artificial oxygen carrier according to claim 6, wherein the enzyme species is methemoglobin.

9. The artificial oxygen carrier according to claim 4, wherein a membrane constituting the lipid vesicle is modified.

10. The artificial oxygen carrier according to claim 9, wherein the membrane is modified with polyethylene glycol.

11. The artificial oxygen carrier according to claim 4, wherein, when the lipid vesicle, encapsulating an agent for preventing methemoglobin formation and a hemoprotein, is at 37° C. under a partial pressure of oxygen of between 5 and 300 Torr for 60 hours, the rate of methemoglobin is 50% or less.

12. The artificial oxygen carrier according to claim 4, wherein, when hydrogen peroxide is added to the lipid vesicle, encapsulating an agent for preventing methemoglobin formation and a hemoprotein, and when the mixture is left for 60 minutes, the rate of methemoglobin is 20% or less.

13. A method comprising:
encapsulating an agent containing tyrosine that prevents methemoglobin formation and a hemoprotein in a lipid vesicle for producing an artifical oxygen carrier.

14. A method comprising:
encapsulating an agent containing tyrosine that prevents methemoglobin formation and a hemoprotein in a lipid vesicle for preventing methemoglobin from the hemoprotein.

15. A method comprising:
encapsulating an agent containing tyrosine that prevents methemoglobin formation and a hemoprotein in a lipid vesicle for storing an artificial oxygen carrier.

16. The method according to claim 13, wherein the hemoprotein is hemoglobin.

17. The method according to claim 13, which further comprises encapsulation of enzyme species in the lipid vesicle.

18. The method according to claim 17, wherein the enzyme species is catalase.

19. The method according to claim 17, wherein the enzyme species is methemoglobin.

20. The method according to claim 13, wherein, when the lipid vesicle, encapsulating an agent for preventing methemoglobin formation and a hemoprotein, is at 37° C. under a partial pressure of oxygen of between 5 and 300 Torr for 60 hours, the rate of methemoglobin is 50% or less.

21. The method according to claim 13, wherein, when hydrogen peroxide is added to the lipid vesicle, encapsulating an agent for preventing methemoglobin formation and a hemoprotein, and when the mixture is left for 60 minutes, the rate of methemoglobin is 20% or less.

22. An artificial oxygen carrier comprising:
a lipid vesicle encapsulating an agent containing tyrosine for preventing methemoglobin formation, a hemoglobin, and catalase or methemoglobin,
wherein a membrane of the lipid vesicle is modified with polyethylene glycol.

23. The method according to claim 14, wherein the hemoprotein is hemoglobin.

24. The method according to claim 14, which further comprises encapsulation of enzyme species in the lipid vesicle.

25. The method according to claim 24, wherein the enzyme species is catalase.

26. The method according to claim 24, wherein the enzyme species is methemoglobin.

27. The method according to claim 14, wherein, when the lipid vesicle, encapsulating an agent for preventing methemoglobin formation and a hemoprotein, is at 37° C. under a partial pressure of oxygen of between 5 and 300 Torr for 60 hours, the rate of methemoglobin is 50% or less.

28. The method according to claim 14, wherein, when hydrogen peroxide is added to the lipid vesicle, encapsulating an agent for preventing methemoglobin formation and a hemoprotein, and when the mixture is left for 60 minutes, the rate of methemoglobin is 20% or less.

29. The method according to claim 15, wherein the hemoprotein is hemoglobin.

30. The method according to claim 15, which further comprises encapsulation of enzyme species in the lipid vesicle.

31. The method according to claim 30, wherein the enzyme species is catalase.

32. The method according to claim 30, wherein the enzyme species is methemoglobin.

33. The method according to claim 15, wherein, when the lipid vesicle, encapsulating an agent for preventing methemoglobin formation and a hemoprotein, is at 37° C. under a partial pressure of oxygen of between 5 and 300 Torr for 60 hours, the rate of methemoglobin is 50% or less.

34. The method according to claim 15, wherein, when hydrogen peroxide is added to the lipid vesicle, encapsulating an agent for preventing methemoglobin formation and a hemoprotein, and when the mixture is left for 60 minutes, the rate of methemoglobin is 20% or less.
